(19) United States
(12) Patent Application Publication
(10) Pub. No.: US 2012/0283332 A1
(43) Pub. Date: Nov. 8, 2012

(54) TRANSDERMAL DELIVERY OF METFORMIN

(75) Inventors: Chase A. Scarbrough, Findlay, OH (US); Stanley S. Scarbrough, Findlay, OH (US); Jay Shubrook, Athens, OH (US)

(73) Assignee: OHIO UNIVERSITY, Athens, OH (US)

(21) Appl. No.: 13/504,799

(22) PCT Filed: May 12, 2010
(86) PCT No.: PCT/US10/34542

§ 371 Date: Jul. 16, 2012

Related U.S. Application Data
(60) Provisional application No. 61/177,625, filed on May 12, 2009.

(51) Int. Cl. A61K 31/155; A61P 3/08; A61P 15/08; A61P 3/10

(52) U.S. Cl. 514/635

(57) ABSTRACT

Provided is a transdermal metformin that is an effective alternative treatment modality in patients with insulin resistance. Transdermal metformin can be used in conditions where oral metformin is indicated such as Type 2 diabetes mellitus, pre-diabetes, polycystic ovarian syndrome, and other known diabetes associated disorders. One advantage of using transdermal metformin is its ability to bypass the gastrointestinal system. This allows the drug to not have the gastrointestinal side-effects associated with oral metformin. A surprising advantage of using transdermal metformin in accordance with this disclosure is a 90% decrease in dosage from the oral preparation.

### CROSS REFERENCE TO RELATED APPLICATION

This application claims priority to and any other benefit of U.S. 61/177,625, filed May 12, 2009, the entirety of which is incorporated herein by reference.

### BACKGROUND

It is estimated that approximately 23.6 million individuals in the United States have diabetes mellitus, 90% of which have Type 2 diabetes mellitus. This accounts for 8% of the population and that number is expected to double by the year 2025. Americans spend $113 billion dollars a year in the management of diabetes. A diagnosis of diabetes also predisposes to conditions such as hypertension, hyperlipidemia, myocardial infarction and stroke.

Type 2 diabetes mellitus is described as impaired insulin secretion, insulin resistance, excessive hepatic glucose production, and abnormal fat metabolism. Obesity is commonly seen in type 2 diabetes. Early in the development of type 2 diabetes, the insulin producing pancreatic beta cells are able to compensate for insulin resistance by increasing the amount of insulin release. As diabetes progresses, the pancreatic islets are unable to maintain the hyperinsulinemic state, characterized by elevated postprandial glucose levels and peripheral insulin resistance. Beta cell exhaustion leads to a decline in insulin secretion and increased hepatic glucose production. Ultimately, type 2 diabetes progresses to elevated fasting glucose levels.

Metformin is considered a first-line treatment in the management of insulin resistance and type 2 diabetes mellitus. Metformin is often used to decrease fasting and postprandial hyperglycemia in combination with modifications to the diet and lifestyle changes. Metformin can also decrease triglycerides levels and has also been associated with a modest weight loss. Metformin has the potential to stimulate lactic acid production when renal excretion is decreased, thus it is contraindicated in patients with renal insufficiency. Likewise, lactic acid production is also a problem in patients with hepatic insufficiency because defective hepatocytes are unable to remove lactate.

Adverse reactions are frequent in patients taking metformin. The most notable are the gastrointestinal side effect such as anorexia, nausea, vomiting, abdominal discomfort and diarrhea. Up to 20% of patients taking oral metformin will experience one of these side effects. The effects are dose related however, up to 5% will discontinue the therapy due to the side effects. 77% of patients taking metformin will also develop a vitamin B12 deficiency. Vitamin B12 deficiency has been associated with peripheral neuropathy, a common finding among type 2 diabetics.

Metformin is absorbed over 6 hours. The bioavailability of oral metformin is only 50-60% under fasting conditions. Food delays the absorption. Gastrointestinal side effects frequently lead patients to not take metformin on an empty stomach, thus reducing the bioavailability.

Importantly, the inventors have discovered that delivery of metformin in the compositions disclosed herein result in an acceptable dose that is in the amount of one tenth ( 1/10) the typical oral dose of metformin. The present invention has certain surprising advantages over the art. By by-passing the gastrointestinal (GI) tract, gastrointestinal complications and side effects of oral formulations of metformin and its salts can be avoided. In known formulations that are ingested, a higher amount of pharmaceutical agent is required per dose due to the problem of degradation in the GI tract. The present compositions which deliver the pharmaceutical agent through the skin are formulated with active ingredient to be provided in a significantly reduced actual dose to achieve clinically comparable effects in comparison to the typical oral dose. This leads to significantly reduced side effects associated with oral delivery, improved compliance and tolerability for patients who have difficulty swallowing oral agents, and cost savings.

### SUMMARY

Provided are topical metformin compositions for transdermal delivery of metformin to an animal, the compositions comprising a pharmaceutically acceptable carrier and an effective amount of a pharmaceutical agent comprising metformin or a pharmaceutically acceptable salt thereof contained in said carrier, said carrier being capable of delivering a pharmaceutically effective amount of said pharmaceutical agent for transdermal absorption.

Also provide is a process for making the topical metformin compositions for transdermal delivery, the process comprising mixing an effective amount of a pharmaceutical agent consisting of metformin or a pharmaceutically acceptable salt thereof with an effective amount of at least one absorption enhancer and pharmaceutically acceptable salts and analogues thereof to form a preparation. The preparation is combined with a suitable pharmaceutically acceptable carrier to make the present composition.

Also provided is a method of using, and a use of, the topical metformin compositions for transdermal delivery to treat various conditions chosen from diabetes, pre-diabetes, polycystic ovarian disease, and known diabetes associated disorders. The present composition can be useful to decrease plasma glucose levels, decrease hepatic glucose production, decrease lipid levels, increase sensitivity to insulin, decrease intestinal absorption of glucose, and decrease hypoglycemia. The method involves administering to a subject the topical metformin composition for transdermal delivery in order to treat such conditions. The invention also provides a use of the composition in the manufacture of a medicament for treating the same conditions. The composition can be maintained on the skin for at least six hours before washing or swimming.

### DETAILED DESCRIPTION

The present invention will now be described with occasional reference to the specific embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

In various embodiments, compositions are provided comprising anti-diabetes therapeutic agents, namely biguanides (i.e., metformin) for transdermal delivery. Transdermal metformin may be introduced into the body using compositions comprising metformin and one or more penetration enhancers.

According to some embodiments, the metformin may be in the form of one or more pharmaceutically acceptable salts thereof (e.g. metformin hydrochloride, N,N-dimethylimidodicarbonimidic diamide hydrochloride). In some examples, the compositions also comprise pluronic lecithin organogel (PLO). In other examples, the penetration enhancer may be selected from dimethylsufloxide, lecithin, lecithin isopropyl palmitate and one or more of an alkali metal alkyl sulfate, glycerin, a bile acid or bile salt, hyaluronic acid, octylphenoxypolyethoxyethanol, glycolic acid, lactic acid, chamomile extract, cucumber extract, oleic acid, linolenic acid, borage oil, evening primrose oil, polyglycerin, lysine, polylysine, triolein, monoolein, monooleates, monolaurates, menthol, polidocanol alkyl ethers, chenodeoxycholate, deoxycholate and pharmaceutically acceptable salts and analogues thereof. In yet other examples, the penetration enhancers may comprise PLO together with one or more additional penetration enhancers selected from those described above, or others known in the art.

The penetration enhancer known as PLO is a biphasic compound consisting of a water phase and a lipid phase. In some examples, the lipid phase is prepared by mixing isopropyl palmitate and lecithin, and the water phase is prepared by mixing pluronic (a group of surfactants comprising block copolymers based on ethylene oxide and propylene oxide that can function as antifoaming agents) and water. The two mixtures are then added together through high agitation to create one standing compound. Metformin can be directly added to the compound at this point or can be added during the preparation of the aqueous phase.

In clinical use of metformin formulated with PLO, it was surprisingly observed that transdermal delivery of approximately 5-10% of the amount of the oral dose elicited a therapeutic response in terms of decreased blood-glucose levels in the transdermal preparations of metformin. For example, if a patient was taking 1000 mg of metformin by mouth twice a day, an equivalent transdermal preparation would be 100 mg transdermally twice a day. This can be achieved by the patient applying as little as 0.5 ml (50 mg) of the transdermal metformin gel to the skin twice a day. Thus, the amount of the metformin containing pharmaceutical agent administered according to the instant compositions can be from about 50 to 200 milligrams, as compared to daily oral metformin doses that can be as much as 1000 mg or more. Good results have been obtained at a dosage of 100 mg daily delivered transdermally according to the instant compositions to achieve the same effective dose as achieved for a patient taking about 1000 mg orally.

The daily dosage of transdermally delivered metformin in accordance herewith will be between 5 and 500 mg per daily dose. Thus, the dose of metformin may be from about 5, 6, 7, 8, 9, 10, 20, 30, 40, 50, 60, 70, 80, 90, 100, 200, 300, 400 and 500 mg and increments therebetween.

It will be appreciated by one skilled in the art that an effective amount of the transdermal metformin composition is an amount sufficient to bring about the desired result, such as obtaining the intended therapeutic treatment or prevention of a disorder in a patient, or regulating a physiological condition in a patient, as further described herein. Such an amount will therefore be understood as having a therapeutic and/or prophylactic effect in a patient. Also as described herein, it will be appreciated that the effective amount will vary depending on the specific patient, and that the effective dose does not predictably vary based on the patient's weight or other physical property, but will vary based on the nature and severity of the disorder being treated, the patient being treated, and the characteristics of the combination of absorption enhancers used.

Accordingly, in determining the effectiveness of treatment, it will be understood that any one or more of the following are typically associated with clinically significant improvement in a patient, including: decrease in one or more of blood glucose levels, hepatic glucose production, and lipid levels, or decrease in intestinal absorption of glucose, or decrease hypoglycemia or other clinically significant changes can be therapeutic and/or prophylactic as can be any increase in sensitivity to insulin. In some examples, improvements may be seen in one or more of the following indicators using testing methods well known in the art for testing blood/serum levels of indicators, and consistent with the results reported in the examples herein, including: reduction in blood sugar; reduction in hemoglobin A1C; minimal reduction in blood levels of vitamin B12 as compared to treatment with oral metformin; reduction in triglycerides; improvement of liver function, as evidenced by decrease in one or more of aspartate transaminase (AST) and alanine transaminase (ALT); and reduction in microalbumin.

Thus, in some examples, topical administration of metformin in accordance with the disclosure will result in clinical effectiveness as reflected by a reduction in the patient's blood glucose by about at least 10% to about at least 40% as compared to the patient's blood glucose level measured prior to administration of transdermal metformin. Accordingly, with respect to the blood glucose indicator, a clinical significant reduction, comparable to oral dosages of metformin that are up to 90% or greater than the instant method, will be at least 10, 11, 12, 13, 14, 15, 16, 17, 18, 19, 20, 25, 30, 35, 40% or more, and the decrease may be greater than 40, 45, 50, 55, 60, 65, 70, 75 or 80%.

The precise dosage level of the instant compositions should be determined by the attending physician or other health care provider. It will further be appreciated that transdermal delivery may be achieved in one or multiple doses within the range from about 5 to about 500 mg of metformin per day, per patient. Of particular importance with respect to the invention hereof is that significantly lower doses of metformin have been shown to be at least as effective as traditionally delivered oral dosages, where the difference is as much as 90% or greater.

Thus, typically, the present compositions will contain about 5 to 500 milligrams per daily dose. Depending on the dosing regimen, each dose can contain 5 to 500 mg, and can be delivered in multiple doses, as needed, to achieve the prescribed daily dose. It will be appreciated that the amount may vary slightly depending on, amongst other things, the release characteristics of the carrier employed and the presence of any other agents that may influence delivery rate. Moreover, the number of doses per day and the amount of agent per dose may be selected to achieve the best compliance of the patient. Good results have been obtained with a daily dose of 100 mg, delivered in two separate doses of 50 mg each.

Each dose can contain from about 5 to 90, or from about 10 to 80 w/w %, or from about 20 to 80 or 20 to 50 w/w % of metformin based on the total weight of the composition, depending upon the amount of the penetration enhancer and any other carrier or optional additive present.

The compositions described herein may optionally be formulated in a suitable gel or ointment containing the active component suspended or dissolved in one or more carriers. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Carriers for topical administration of the compositions of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable gel, lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

It will be appreciated by one of skill in the art that the topical compositions hereof may optionally contain additional ingredients, that may provide additional and possibly different therapeutic benefits. The compositions may also optionally contain one or more color, scent, or preservative enhancers.

The following examples describe representative embodiments of formulations and use thereof.

### EXAMPLE 1

Representative Formulation: Metformin 200 mg/ml in 30% PLO

Preparation of 30% pluronic gel: Pluronic gel was prepared as a stock solution for preparing metformin transdermal compositions. The gel was prepared by mixing potassium sorbate (NF Powder, 0.3 gm, PCCA #30-1107) and pluronic F-127 granules (30 gm, (30%) Medisca #2367) and bringing the mixture to a volume of 100 ml with refrigerated purified water. A mixer (BRAUN) was used for mixing. The gel was refrigerated when all of the granules were wet. Dissolution may take place upon cooling and gel will solidify at room temperature and may be returned to gel state by mixing. The resultant ˜100 ml of gel comprised 0.003 gm/ml, or 0.3%, potassium sorbate, and 0.3 gm/ml, or 30% pluronic.

Preparation of lecithin/isopropyl palmitate solution: Lecithin/isopropyl palmitate solution was prepared as a stock for preparing transdermal compositions. The solution was prepared using 100 gm Lecithin Soya granule FCC II powder (PCCA #30-1309), 100 gm liquid isopropyl palmitate (CCA #30-1665), and 0.66 gm sorbic acid NF-FCC powder (Medisca #0527). The lecithin and sorbic acid were dispersed in the isopropyl palmitate (where 100 gm=117 ml), and allowed to stand overnight. A liquid of syrup consistency was formed, and owing to its color and viscosity, is sometimes referred to as “motor oil.” The mixture was mixed by hand to wet all lecithin granules and is stored covered. The resultant ˜220 ml of solution comprised 0.455 gm/ml or 45.5% lecithin, 0.455 gm/ml or 45.5% isopropyl palmitate, and 0.003 gm/ml. or 0.3% sorbic acid.

Preparation of Metformin PLO gel: Powdered metformin hydrochloride (approximately 6 gm, PCCA #30-4400) was mixed with 7.7 ml of lecithin/isopropyl palmitate solution and 30 ml of 30% pluronic gel and mixed until even consistency was achieved. In some examples, Lipoderm (PCCA#30-3338)or similar agent may be added to maintain consistency, especially in cold weather conditions. The resultant composition was dispensed in appropriate aliquots for topical administration in amounts of from about 0.1 ml to up to 2.5 ml or more per dose of the 200 mg/ml gel, as prescribed by the clinician.

### EXAMPLE 2

Representative Formulation: Metformin 200 mg/ml in 20% PLO

Preparation of 20% pluronic gel: Pluronic gel may be prepared in an alternate formulation of 20% according to the method described above, using 20 gm of pluronic F-127 ((20%) Medisca #2303) and preparing and combining all other ingredients as described above.

### EXAMPLE 3

Representative Formulation: Metformin 100 mg/ml in DMSO/10% PLO

In yet other embodiments, metformin may be combined with the penetration enhancer DMSO and PLO. In one example, dimethylsulfoxide (6 ml Medisca #2430) is mixed with 13.2 ml lecithin/isopropyl palmiatat soution and 60 ml of 20% pluroonic gel. All ingredients are measured and mixed in a beaker, milled to uniform consistency, and stored in a tube as a stock solution for combination with metformin The resultant ˜60 ml of solution will comprise 0.22 ml or 22% lecithin/isopropyl palmitate, and 0.1 gm/ml or 10% pluronic and 10% DMSO.

Preparation of metformin/DMSO/20% PLO gel. Powdered metformin (6 gm) is mixed with 60 ml of DMSO 10% PLO described above. The resultant composition may be dispensed in appropriate aliquots for topical administration in amounts of from about 0.2 ml to up to 5 ml or more per dose of the 100 mg/ml gel, as may be prescribed by the clinician.

### EXAMPLE 4

### Clinical Case Report 1

History of chief complaint: A 33-year-old biracial female presented for follow-up clinical evaluation. She reported that her blood sugar readings were routinely 242-425 mg/dL. When she had high blood sugar readings she gas associated nausea. She did not have any lows. She had taken in the past metformin but she did not tolerate high doses. She took low dose pramlintide (Symlin®, Amylin & Lilly, Indianapolis, Ind.) and it did not make her sick but stopped it due to the number of injections.

Past medical history: DM2 (diagnosed 6 years before). Elevated LFT's from non-alcoholic steatohepatitis, Dyslipidemia.

Social Hx: Unemployed, no alcohol, tobacco or recreational drug use.

Meds: Asa, Fish Oils, glargine (Lantus®, Bridgewater, NJ) 55 units every evening, omeprazole 20 mg twice a day, novolog 20 units before meals, sertraline 100 mg daily, naproxyn 500 mg three times a day, gabapentin 150 mg twice a day.

Family history: Mother: DM2, HTN, Type IV Hyperlipidemia, DM2 ESRD on Dialysis, Amputation. Father: CVAx2; MIx3; hyperlipidemia; HTN; DM2

Review of systems: Pertinent positives/negatives include: cardiovascular: denies chest pain, SOB, palpitations, or orthopnea; gastrointestinal: some stomach discomfort, denies nausea, vomiting, diarrhea, constipation; renal: microalbumin; positive 2006, negative Apr. 26, 2008. Not on ACE therapy due to child-bearing status; endocrine: no significant change in weight.

Vitals: HT. 5′5″ ft; WT. 200.2 LBS; BMI 30; BP: 118/82 mm/hr

Labs: BS: 361, Hgb A1C 10.7%, ALT: 104, AST: 55, Alkaline Phosphatase: 214, Total Cholesterol: 206, Triglycerides: 421, HDL: 27, unable to calculated LDL when triglycerides are above 400.

The patient had stopped taking metformin 3 months prior to visit due to severe gastrointestinal side effects. Metformin had improved her BS readings. Since going off metformin, she reported that her BS readings were between 250-350 mg/dL with no lows. Clinical judgment was made that the patient would benefit from an alternative drug delivery mechanism. The patient was started on transdermal metformin gel 100 mg/ml, apply 0.5 ml topically to the wrist twice a day. The patient was counseled on how to use the medication and potential side effects.

On follow-up after two months of use on the transdermal metformin gel she was doing better overall. While using transdermal metformin in combination with her insulin she saw her FSG running below 150 mg/dL. The patient did not have any reactions or adverse side effects associated with transdermal metformin preparation. The patient also had a slight drop in her Hgb A1C to 10.1%, down from 10.7% two months prior. The patient saw no change in her LFTs with her ALT was 110, AST 60 and Alkaline Phosphatase 145.

In this particular case, we also observed a decrease in the patient's blood glucose readings by an average of 250-300 mg/dL over a 7 month period with a dosage of transdermal metformin between 100-200 mg per day. There was a short time period where the patient did not have any transdermal metformin and she experienced a drastic spike in her blood-glucose readings to the 500-600 mg/dL range. Upon restarting the transdermal metformin, her blood-glucose reading returned to previous levels. This patient also appreciated a decrease in her Hgb A1C by 0.4%, as well as a decrease in her total cholesterol, triglycerides and LDL levels without any other change in the patient's medications or lifestyle modifications.

### EXAMPLE 5

### Clinical Case Report 2

History of chief complaint: A 38-year-old biracial female presented for routine follow-up of her Type 2 diabetes. Her diabetes was uncontrolled. She checked her BS 2× a day and she was running 400-500 mg/dL. She had tried metformin, glumetza, pioglitazone (Actos®, Takeda, Tokyo, Japan), exenatide (Byetta®, Amylin & Lilly, Indianapolis, IN), sitagliptin (Januvia®, Merck, Whitehouse Station, N.J.) and multiple sulfonylureas but had been unable to tolerate any of them. She had recently stopped all diabetic medications due to side effects. She wished to stay off insulin due to the risk of gaining weight.

Past medical history: DM2, hyperlipidemia, multinodular goiter with dominant nodule.

Social Hx: No alcohol, tobacco or recreational drug use.

Surgeries: C-Sections×3

Meds: lisinopril, simvastatin

Family history: Mother: DM2, HTN, Type IV Hyperlipidemia, goiters, diabetic nephropathy & retinopathy. Father: DM2, HTN, Type IV Hyperlipidemia.

Review of systems: Pertinent positives/negatives included: Cardiovascular: denies chest pain, SOB, palpitations, or orthopnea; Gastrointestinal: denies abdominal pain, nausea, vomiting, diarrhea, constipation. Oral anti-diabetic medications gave her an upset stomach and constipation. Gets reflux frequently; Renal: microalbumin; positive ×1; Endocrine: no significant change in weight.

Vitals: P: 82 HT. 5′5″ ft; WT. 126.4 LBS; BMI 21; BP: 121/62 mm/hg

No physical examination abnormalities appreciated.

Labs: Hgb A1C 11.2%, Total Cholesterol: 159, Triglycerides: 284, HDL: 32, LDL: 70

The patient was not on any diabetic medications due to intolerable gastrointestinal side effects. Her BS were running between 400-500 mg/dL. It was decided that an alternative drug delivery mechanism would be appropriate. The patient was started on a transdermal metformin gel. The patient was educated on the usage of the gel, how to apply the gel and potential side effects. The patient was to contact the physician if any adverse effects occurred. An initial dosage of transdermal metformin gel 100 mg/ml, apply 0.5 ml topically to the wrists twice a day was order and started. The patient was to follow-up in one month.

On follow-up a month later, the patient stated having no side effects to transdermal metformin. Her BS were running in the mid 200's without any lows. The patient stated that there had been no lifestyle changes. Hgb A1C was 10.8%. The patient's Type 2diabetes was uncontrolled but improving. An increase of transdermal metformin was made to 200 mg/ml, apply 0.5 mg topically to the ventral wrists twice a day.

On 3 month follow-up of using transdermal metformin the patient stated that her FSG were in the 200's. She reported that she is having no side effects and denied any other problems. Lipids were collected with results of total cholesterol: 119, triglycerides: 209, HDL: 29, LDL: 48. This was an improvement from her previous levels. CBC, TSH and CMP were within normal limits.

The patient was seen for a 5 month follow-up and expressed some concern that she was having elevated sugars in the evening after dinner. She was interested in trying a medication that will cover her through the evening to get her BS to 200 mg/dL in the morning. She was concerned with anything that will cause her to gain weight. Exenatide 5 mcg was added to her regimen only before dinner to help with her post dinner sugar.

The patient was seen for a 7 month follow-up. The patient was out of the transdermal metformin and had not reordered the gel. After stopping the transdermal metformin her BG increased to the 500-600 mg/dL range even while increasing her exenatide dose to twice a day. Upon restarting transdermal metformin her BG returned to the 300-400 mg/dL range after about a week.

In this particular case, we observed a decrease in the patient's blood glucose readings by an average of 250-300 mg/dL over a 7 month period with a dosage of transdermal metformin between 100-200 mg per day. There was a short time period where the patient did not have any transdermal metformin and she experienced a drastic spike in her blood-glucose readings to the 500-600 mg/dL range. Upon restarting the transdermal metformin, her blood-glucose reading returned to previous levels. This patient also appreciated a decrease in her Hgb A1C by 0.4%, as well as a decrease in her total cholesterol, triglycerides and LDL levels without any other change in the patient's medications or lifestyle modifications.

The foregoing detailed description of various representative embodiments show by way of description and illustration, and not by way of limitation, representative embodiments. It is to be understood that other embodiments are contemplated though not depicted or described herein, and that logical, mechanical, chemical and electrical changes may be made without departing from the spirit and scope of the present invention. In this patent document, the word “comprising” is used in its non-limiting sense to mean that items following the word are included, but items not specifically mentioned are not excluded. Thus, the terms “comprising”, “having”, “including”, and “containing” are to be construed as open-ended terms (i.e., meaning “including, but not limited to”). A reference to an element by the indefinite article “a” does not exclude the possibility that more than one of the element is present, unless the context clearly requires that there be one and only one of the elements.

Unless otherwise defined, all technical and scientific terms used herein have the same meaning as commonly understood by one of ordinary skill in the art to which this invention belongs. The terminology used in the description herein is for describing particular embodiments only and is not intended to be limiting. As used in the description and the appended claims, the singular forms “a,” “an,” and “the” are intended to include the plural forms as well, unless the context clearly indicates otherwise. All publications, patent applications, patents, and other references mentioned herein are expressly incorporated by reference in their entirety.

Unless otherwise indicated, all numbers expressing quantities of components, reagents, ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, any numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Other embodiments will be apparent to those skilled in the art from consideration of the specification and practice of the disclosure. It is intended that the specification and examples be considered as exemplary only, and that modifications may be made to the described embodiments without departing from the spirit and scope as defined herein.

### REFERENCES

Masharani Umesh, “Chapter 27. Diabetes Mellitus & Hypoglycemia” (Chapter). McPhee S J, Papadakis M A, Tierney L M, Jr.: CURRENT Medical Diagnosis & Treatment 2010: URL: accessmedicine.com/content.aspx?aID=15524.

University of Chicago Medical Center (2009, Nov. 27). Diabetes cases to double and costs to triple by 2034. ScienceDaily. Retrieved Apr. 16, 2010, from URL: sciencedaily.com/releases/2009/11/091127101038.htm

Rush Micheal D, Winslett Sonia, Wisdom Kimberley D, “Chapter 212. Diabetes Mellitus” (Chapter). Tintinalli J E, Kelen G D, Stapczynski J S, Ma O J, Cline D M: Tintinalli's Emergency Medicine: A Comprehensive Study Guide, 6e: URL: accessmedicine.com/content.aspx?aID=606123.

Powers Alvin C, “Chapter 338. Diabetes Mellitus” (Chapter). Fauci A S, Braunwald E, Kasper D L, Hauser S L, Longo D L, Jameson J L, Loscalzo J: Harrison's Principles of Internal Medicine, 17e: URL: accessmedicine.com/content.aspx?aID=2891108.

Clinical Guidelines Task Force, International Diabetes Federation (2005). “Glucose control: oral therapy”. In: Global Guideline for Type 2 Diabetes. Brussels: International Diabetes Federation, 35-8. Retrieved on Apr. 16, 2010.

Masharani Umesh, German Michael S, “Chapter 18. Pancreatic Hormones & Diabetes Mellitus” (Chapter). Gardner D G, Shoback D: Greenspan's Basic and Clinical Endocrinology, 8e: URL: accessmedicine.com/content.aspx?aID=2633151.

DrugBank. (2009, Apr. 19). Showing drug card for metformin. Retrieved Apr. 16, 2010 from URL: drugbank.ca/drugs/DB00331.

Murdan, S. (2005). A review of pluronic lecithin organogel as a topical and transdermal drug delivery system. Hospital Pharmacist, 12,267-270.

Kumar, R., Katare O. P. (2005). Lecithin Organogels as a Potential Phospholipid-Structured System for Topical Drug Delivery: A Review. AAPS PharmSciTech, 6(2) Article 40, E298-E310.

Franckum, J., Ramsay, D., Das, N. G., Das, S. K. (2004). Pluronic Lecithin Organogel for Local Delivery of Anti-Inflammatory Drugs. International Journal of Pharmaceutical Compounding, 8(2), 101-105.

Warner, J. (2009, June 8). Metformin linked to B12 Deficiency. Retrieved Apr. 20, 2010 from URL: medicinenet.com/script/main/art.asp?articlekey=101042.

1. A method for treating diabetes in a patient: comprising administering to the patient an effective amount of metformin, or a salt thereof, in a composition formulated for topical administration and comprising the metformin and at least one penetration enhancer selected from pluronic lecithin organogel (PLO), dimethylsufloxide, lecithin, lecithin isopropyl palmitate and one or more of an alkali metal alkyl sulfate, glycerin, a bile acid or bile salt, hyaluronic acid, octylphenoxypolyethoxyethanol, glycolic acid, lactic acid, chamomile extract, cucumber extract, oleic acid, linolenic acid, borage oil, evening primrose oil, polyglycerin, lysine, polylysine, triolein, monoolein, monooleates, monolaurates, menthol, polidocanol alkyl ethers, chenodeoxycholate, deoxycholate and pharmaceutically acceptable salts and analogues thereof.

2. The method, according to claim 1, wherein the effective amount of the metformin composition is administered to the patient is about 5 to 500 mg per day.

3. The method according to claim 2, wherein the metformin composition is administered in multiple doses per day.

4. The method according to claim 2, wherein the effective amount of the metformin composition is about 100 to 200 mg per day, administered in at least two doses of approximately equal amount.

5. The method according to claim 1, wherein the effective amount of metformin is selected as a percentage of a standard oral dose selected from the standard oral dose used by the patient and the standard oral dose of about 1000 mg/day.

6. The method according to claim 5, wherein an initial dose is about 10% to about 100% of the selected standard dose.

7. The method according to claim 6, wherein the initial dose is about 10% to about 30% of the selected standard dose.

8. The method according to claim 7, wherein the absorption enhancer is PLO present in amount that is from about 10% to about 40% v/v.

9. The method according to claim 6, wherein the composition comprises at least a second absorption enhancer.

10. The method according to claim 9, wherein the second absorption enhancer is DMSO present in an amount that is about 10% v/v.

11. The method of claim 1, wherein the effective amount of metformin administered to the patient reduces the patient's blood glucose by about at least 10% to about at least 40% as compared to the patient's blood glucose level measured prior to administration of transdermal metformin.

12. A method of decreasing the blood glucose level, decreasing hepatic glucose production, decreasing lipid levels, increasing sensitivity to insulin, decreasing the intestinal absorption of glucose, or decreasing hypoglycemia in a subject: comprising administering to the subject a topical metformin composition comprising an effective amount of a pharmaceutical agent comprising metformin, or a pharmaceutically acceptable salt thereof and at least one absorption enhancer chosen from pluronic lecithin organogel (PLO), dimethylsufloxide, lecithin, lecithin isopropyl palmitate and one or more of an alkali metal alkyl sulfate, glycerin, a bile acid or bile salt, hyaluronic acid, octylphenoxypolyethoxyethanol, glycolic acid, lactic acid, chamomile extract, cucumber extract, oleic acid, linolenic acid, borage oil, evening primrose oil, polyglycerin, lysine, polylysine, triolein, monoolein, monooleates, monolaurates, menthol, polidocanol alkyl ethers, chenodeoxycholate, deoxycholate and pharmaceutically acceptable salts and analogues thereof.

13. The method of claim 12, wherein the subject is at risk for or is known to have diabetes.

14. The method according to claim 13, wherein administration of the metformin composition decreases the subject's blood levels of one or more of blood sugar, hemoglobin Al C, and tryglycerides, aspartate transaminase, alanine transaminase, and microalbumin.

15. The method according to claim 13, wherein administration of the metformin composition increases the subject's sensitivity to insulin.

16. The method according to claim 13, wherein administration of the metformin composition decreases the subject's intestinal absorption of glucose.

17. The method according to claim 13, wherein administration of the metformin composition decreases the subject's hypoglycemia.

18. The method according to claim 13, wherein administration of the metformin composition does not significantly affect a reduction in the patient's blood levels of vitamin B12 relative to those levels prior to administration of metformin.

19. A transdermal metformin gel composition, comprising:
metformin or a pharmaceutically acceptable salt thereof;
lecithin;
isopropyl palmitate; and
one or more ethylene oxide/propylene oxide block copolymers,
wherein the metformin or a pharmaceutically acceptable salt thereof is present in the transdermal metformin gel composition in an amount of from about 5 w/w% to about 90 w/w%.

20. The transdermal composition of claim 19, wherein the metformin or a pharmaceutically acceptable salt thereof is present in the transdermal metformin gel composition in an amount of from about 10 w/w% to about 80 w/w%.

21. The transdermal composition of claim 19, wherein the metformin or a pharmaceutically acceptable salt thereof is present in the transdermal metformin gel composition in an amount of from about 5 w/w% to about 50 w/w%.

22. The transdermal composition of claim 19, wherein the metformin or a pharmaceutically acceptable salt thereof is present in the transdermal metformin gel composition in an amount of from about 5 w/w% to about 20 w/w%.

23. The transdermal composition of claim 19, wherein the metformin or a pharmaceutically acceptable salt thereof is present in the transdermal metformin gel composition in an amount of from about 10 w/w% to about 20 w/w%.

24. The transdermal composition of claim 19, wherein the metformin or a pharmaceutically acceptable salt thereof is present in the transdermal metformin gel composition in an amount of from about 10 w/w% to about 50 w/w%.

25. The transdermal composition of claim 19, wherein the metformin or a pharmaceutically acceptable salt thereof is present in the transdermal metformin gel composition in an amount of from about 50 mg/ml to about 200 mg/ml.

26. The transdermal composition of claim 19, wherein metformin is present in the transdermal metformin gel composition in an amount of from about 100 mg/ml to about 200 mg/ml.

27. A method for treating diabetes mellitus, pre-diabetes, polycystic ovarian syndrome, or insulin resistance, in a patient, comprising:
topically administering to the patient, a therapeutically effective amount of the composition of claim 19.

28. A method for treating diabetes mellitus, pre-diabetes, polycystic ovarian syndrome, or insulin resistance, in a patient, comprising: topically administering to the patient, a therapeutically effective amount of transdermal metformin gel composition, comprising: metformin or a pharmaceutically acceptable salt thereof; lecithin; isopropyl palmitate; and one or more ethylene oxide/propylene oxide block copolymers,
wherein the metformin or a pharmaceutically acceptable salt thereof is present in the transdermal metformin gel composition in an amount of from about 10 w/w% to about 50 w/w%.

29. The method of claim 28, wherein the metformin or a pharmaceutically acceptable salt thereof is present in the transdermal metformin gel composition in an amount of from about 10 w/w% to about 20 w/w%.

30. The method of claim 28, wherein the metformin or a pharmaceutically acceptable salt thereof is present in the transdermal metformin gel composition in an amount of from about 50 mg/ml to about 200 mg/ml.
